(19) United States
(12) Patent Application Publication
(10) Pub. No.: US 2014/0163360 A1
(43) Pub. Date: Jun. 12, 2014

(54) IRRIGATED CATHETER

(71) Applicant: Boston Scientific Scimed, Inc., Maple Grove, MN (US)

(72) Inventors: Debbie Stevens-Wright, North Andover, MA (US); David MacAdam, Millbury, MA (US); Charles A. Gibson, Malden, MA (US)

(73) Assignee: Boston Scientific Scimed, Inc., Maple Grove, MN (US)

(21) Appl. No.: 13/804,022

(22) Filed: Mar. 14, 2013

Related U.S. Application Data
(60) Provisional application No. 61/734,905, filed on Dec. 7, 2012.
(60) Provisional application No. 61/751,678, filed on Jan. 11, 2013.

(51) Int. Cl. A61B 1/015; A61B 1/00; A61B 1/04; A61B 1/005; A61B 8/12; A61B 8/00; A61B 5/00; A61B 18/14; A61M 25/01

(52) U.S. Cl. CPC A61B 1/015; A61B 18/1492; A61B 1/00087; A61B 1/04; A61M 25/0105; A61B 8/12; A61B 8/4461; A61B 8/445; A61B 5/4836; A61B 5/0066; A61B 5/0084; A61B 8/4483; A61B 1/0057; A61B 1/00126; A61B 1/00165
USPC 600/427; 600/104; 600/439

(57) ABSTRACT

A catheter includes a fluid network for cooling the ablation electrode, surrounding blood and tissue. The fluid network comprises a circumferential channel having an annular shape and fluidly coupled to at least one proximal longitudinal channel configured to conduct fluid along a proximal length of the catheter. The circumferential channel is configured to conduct fluid about at least a circumferential portion of the catheter. The fluid network further comprises a plurality of distal longitudinal channels fluidly coupled to the circumferential channel, with the plurality of distal longitudinal channels being configured to conduct fluid a long a distal length of the catheter. The fluid network occupies a region of the catheter peripheral to a central longitudinal axis of the catheter such that other components of the irrigated catheter, including at least one imaging sensor, is disposed within the central region of the ablation electrode.

### CROSS-REFERENCE TO RELATED APPLICATIONS

The present application claims the benefit under 35 U.S.C. §119(e) of U.S. Provisional Application Ser. No. 61/734,905, filed on Dec. 7, 2012, titled “Irrigated Catheter,” and of U.S. Provisional Application Ser. No. 61/751,678, filed on Jan. 11, 2013, titled “Irrigated Catheter,” each of which is hereby incorporated by reference in its entirety.

### BACKGROUND

The heart is a very complex organ, which relies on both muscle contraction and electrical impulses to function properly. The electrical impulses travel through the heart walls, first through the atria and then the ventricles, causing the corresponding muscle tissue in the atria and ventricles to contract. Thus, the atria contract first, followed by the ventricles. This order is essential for proper functioning of the heart.

In some individuals, the electrical impulses of the heart develop an irregular propagation, disrupting the heart's normal pumping action. The abnormal heartbeat rhythm is termed a “cardiac arrhythmia.” Arrhythmias may occur when a site other than the sinoatrial node of the heart is initiating rhythms (i.e., a focal arrhythmia), or when electrical signals of the heart circulate repetitively in a closed circuit (i.e., a reentrant arrhythmia).

Techniques have been developed which are used to locate cardiac regions responsible for the cardiac arrhythmia, and to disable the short-circuit function of these areas. According to these techniques, an ablation catheter with one or more electrodes is used to apply energy to a portion of the heart tissue in order to ablate that tissue and produce scars which interrupt the reentrant conduction pathways or terminate the focal initiation. To this end, the ablation catheter transmits energy to the tissue adjacent the electrode to create a lesion in that tissue. One or more suitably positioned lesions will typically create a region of necrotic tissue which serves to disable the propagation of the errant impulse caused by the arrythromogenic focus. Ablation is carried out by applying energy to the catheter electrodes. The ablation energy can be, for example, RF, DC, ultrasound, microwave, or laser radiation.

### SUMMARY

One embodiment is directed to a fluid network of a catheter. The fluid network includes circumferential channel having an annular shape and fluidly coupled to at least one proximal longitudinal channel configured to conduct fluid along a proximal length of the catheter. The circumferential channel is configured to conduct fluid about at least a part of a circumferential portion of the catheter. The fluid network also includes a plurality of distal longitudinal channels fluidly coupled to the circumferential channel, the plurality of distal longitudinal channels being configured to conduct fluid along a distal length of the catheter

Another embodiment is directed to a catheter having a fluid network. The catheter includes a handle; a shaft coupled to the handle and an ablation electrode coupled to the shaft. The catheter also includes a fluid network comprising: at least one proximal longitudinal channel configured to conduct fluid along a proximal length of the catheter; a circumferential channel having an annular shape and fluidly coupled to the at least one proximal longitudinal channel, wherein the circumferential channel is configured to conduct fluid about at least a part of a circumferential portion of the catheter; and a plurality of distal longitudinal channels being fluidly coupled to the circumferential channel, the plurality of distal longitudinal channels/configured to conduct fluid along a distal length of the catheter.

Another embodiment is directed to a method of using a catheter to treat tissue. The catheter comprises an ablation electrode and a fluid network at least partially disposed within the ablation electrode. The method includes acts of forming a lesion in the tissue using ablation energy emitted by the ablation electrode, and conducting fluid through the fluid network to cool the ablation electrode and/or the surrounding tissue. The fluid network comprises at least one proximal longitudinal channel configured to conduct fluid along a proximal length of the catheter, a circumferential channel having an annular shape and fluidly coupled to the at least one proximal longitudinal channel, wherein the circumferential channel is configured to conduct fluid about at least a part of a circumferential portion of the catheter, and a plurality of distal longitudinal channels fluidly coupled to the circumferential channel, the plurality of distal longitudinal channels configured to conduct fluid along a distal length of the catheter.

Another embodiment is directed to an irrigated catheter. The catheter includes an ablation electrode, at least one imaging device, an imaging device steering portion coupled to the at least one imaging device and configured to rotate the at least one imaging device, a fluid network configured to conduct fluid along a length of the catheter and occupying a catheter peripheral region that surrounds the imaging device steering portion.

According to another embodiment, a method of using a catheter to treat tissue is disclosed. The catheter includes an ablation electrode, at least one imaging device, and imaging device steering portion coupled to the at least imaging device and configured to rotate the at least one imaging device. The catheter also includes a fluid network configured to conduct fluid along a length of the catheter. The method includes forming a lesion in the tissue using ablation energy emitted by the ablation electrode, conducting fluid through the fluid network to cool the ablation electrode; and imaging the lesion using the at least one imaging device, wherein the fluid network occupies a catheter peripheral region that surrounds the imaging device steering portion.

According to another embodiment an irrigated catheter is provided. The catheter comprises an ablation electrode; at least one imaging device; an imaging device shaft portion coupled to the at least one imaging device; a fluid network configured to conduct fluid along a length of the catheter and occupying a catheter peripheral region that surrounds the imaging device shaft portion.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A illustrates an overview of an ablation catheter system in accordance with some embodiments;

FIG. 1B illustrates an irrigated catheter comprising a fluid network and an ablation electrode, in accordance with some embodiments;

FIG. 2 illustrates a portion of the fluid network of the catheter shown in FIG. 1;

FIG. 3 illustrates fluid network channels at least partially disposed within an ablation electrode of the catheter shown in FIG. 1;

FIG. 4 illustrates the flow of fluid through a portion of the fluid network of the catheter shown in FIG. 1;

FIG. 5 illustrates a circumferential channel of a fluid network of a catheter, in accordance with some embodiments;

FIG. 6 is a cross-sectional view of a fluid network of a catheter, in accordance with some embodiments;

FIGS. 7-8 illustrate a baffle in a circumferential channel of a fluid network of a catheter, in accordance with some embodiments;

FIG. 9 is another view of the fluid network of the catheter shown in FIG. 1;

FIG. 10 is a perspective view of a distal portion of an irrigated catheter, in accordance with some embodiments;

FIG. 11 shows a cross-sectional view of the distal portion of the irrigated catheter shown in FIG. 10, in accordance with some embodiments;

FIG. 12 shows a view of an imaging device steering portion, in accordance with some embodiments;

FIG. 13 illustrates a coupling between an ablation electrode and steering cables of an irrigated catheter, in accordance with some embodiments;

FIGS. 14 and 15 illustrate different views of an irrigated catheter, in accordance with some embodiments;

FIG. 15 illustrates an infusion line of an irrigated catheter, in accordance with some embodiments;

FIG. 16 illustrates a coupling between an ablation electrode and a contoured infusion line, in accordance with some embodiments;

FIGS. 16A and 16B illustrate alternative embodiments of a contoured infusion line;

FIG. 17 illustrates a method of using catheters described herein, in accordance with some embodiments; and

FIG. 18 is a cross-sectional view of a distal portion of another irrigated catheter, in accordance with some embodiments.

### DETAILED DESCRIPTION

Applying energy to tissue with ablation electrodes to ablate tissue heats the electrode(s), surrounding blood, and the tissue. However, to effectively create a lesion in the target tissue (e.g., to treat a cardiac arrhythmia, perform renal denervation etc.), it is important to control temperature at the electrode-tissue interface to avoid unwanted effects such as charring of blood and increase of impedance at the electrode-tissue interface. One way of controlling the temperature at the electrode-tissue interface is to irrigate the ablation electrode with an irrigation fluid such as saline. The irrigation fluid provides convective cooling, which limits the electrode-tissue interface temperature and thereby limits heating of the blood and the formation of coagulum which may lead to an embolic event such as a stroke.

One conventional type of irrigated ablation electrode comprises a closed-loop irrigation system to circulate an irrigation fluid throughout the ablation electrode to cool the ablation electrode. Irrigation fluid enters the ablation electrode from a fluid source, circulates throughout the electrode, and returns to the fluid source. The inventors have recognized that in some closed-loop irrigation systems, heat is conducted to the blood faster than the heat is carried away by the closed-looped irrigation flow, thereby making the blood susceptible to formation of char and coagulum.

Another conventional type of irrigated ablation electrode, commonly referred to as an open irrigation ablation electrode, includes a central fluid reservoir disposed at the center of the irrigated ablation electrode and radial channels fluidly coupled to the central fluid reservoir which allow irrigation fluid in the reservoir to be released through holes in the exterior of the ablation electrode. The inventors have recognized that a central fluid reservoir occupies a significant amount of space at the center of the electrode—space which could be used for other purposes including sensors for measuring physiologic parameters (e.g., lesion depth). The inventors have further recognized that another problem with some conventional open irrigation ablation electrodes having a central fluid reservoir is that the irrigation fluid does not adequately cool the exterior surface of the ablation electrode because such systems generate low irrigation flow velocities and, as a result, require high volumes of fluid to cool the electrode, surrounding tissue, and blood. The inventors have further recognized that the walls separating the surface of the ablation catheter from the central fluid reservoir are relatively thick. For example, in some instances, the thickness of the central fluid reservoir is less than a third of the thickness of the electrode, and the thickness of the walls separating the central fluid reservoir from the exterior surface of the electrode are more than two thirds of the thickness of the electrode. As a result, the thermal mass of the electrode is high relative to that of the fluid being circulated to cool the electrode.

Accordingly, in some embodiments, an irrigated catheter is provided comprising a fluid network at least partially disposed in the jacket of the ablation electrode rather than in a central region of the ablation electrode. That is, the fluid network is at least partially disposed in a region of the ablation electrode peripheral to the central longitudinal axis of the ablation electrode. As such, irrigation fluid flowing through the fluid network flows closer to the exterior surface of the ablation electrode than in conventional ablation electrodes irrigated with fluid flowing through a central fluid reservoir. Because the fluid network occupies a peripheral rather than a central region of the irrigation electrode, in some embodiments, other components of the irrigated catheter (examples of which are described below) may be disposed within the central region of the ablation electrode, thus increasing the capabilities of the irrigated catheter.

The inventors also have recognized that conventional irrigated catheters which release irrigation fluid into a patient's blood stream may release a significant amount of fluid into the blood stream and, as such, it may be advantageous to reduce the amount of irrigation being released into the blood stream (e.g., for patients having kidney failure). The inventors have further recognized that when irrigation fluid exits an ablation electrode at a sufficiently high velocity, the fluid may efficiently carry more heat away from the surface of the ablation electrode, thereby allowing less fluid to be used to cool the blood and tissue surrounding the ablation electrode to a desired level. Accordingly, in some embodiments, the fluid network of an irrigated catheter is configured so that irrigation fluid is released from the ablation electrode at velocities that allow the fluid to quickly carry heat away from the surface of the ablation electrode.

The inventors also have recognized that conventional irrigated catheters which release irrigation fluid into the blood stream may not release fluid uniformly. For example, the exit velocity of fluid may differ depending on the exit opening from which the fluid is exiting. This may lead to non-uniform cooling of the ablation electrode, surrounding blood and tissue. Accordingly, in some embodiments, the fluid network is configured such that fluid is released from each of multiple exit openings of the ablation electrode at approximately and/or substantially the same exit velocities.

The inventors have also recognized that three-dimensional visualization of ablation lesion formation, catheter contact, and target tissue geometry (e.g., wall thickness) may help to create adequate ablation lesions during the treatment of atrial fibrillation and other arrhythmias. Assessment of transmurality in real time can be helpful in preventing esophageal fistula, bronchial fistula, and over treatment. The inventors have further appreciated that creation of a three-dimensional image of lesion formation can be facilitated by arrangements that allow for a high-resolution assessment of the target ablation site at extremely fast imaging speeds.

The inventors have also recognized that some conventional ablation techniques fail to provide adequate capability for real-time assessment of lesion formation at the target ablation site. Conventional ablation techniques control lesion formation during an ablation procedure based on analysis of initial ablation parameter settings and IECG signals, catheter thermocouple readings, power utilization information, or impedance. However, each of these sources of information has limitations when applied to assessment of the target ablation site. For example, thermocouple recordings reflect the temperature information within the immediate vicinity of the thermocouple itself and, as a result, detailed (e.g., high-resolution three-dimensional) temperature information about the targeted ablation site cannot be obtained from thermocouple recordings. The impedance information of the system reflects the gross impedance of the biological system that lies between the catheter electrode and the reference electrode and does not provide detailed information about the target ablation site. Power utilization is also gross information about the system and does not provide detailed information about the target ablation site. IECG signals may contain electrical information about the target ablation site as well as far field information, but IECG signals do not allow for high resolution sampling of other physiologic information about the target ablation site.

Other techniques for real-time assessment of lesion formation may include optical coherence tomography, magnetic resonance imaging, and ultrasound. However, conventional ablation catheters utilizing these technologies are not well suited for incorporation with other catheter components used for steering, ablation, and temperature sensing. Conventional ablation catheters may not provide sufficient space to provide for such imaging capability (e.g., there may not be sufficient space to provide for a rotatable drive shaft to spin the imaging element). For example, conventional open irrigation ablation electrodes, described above, have insufficient space to implement such imaging technology.

Accordingly, in some embodiments disclosed herein, an irrigated catheter is provided with a layout that allows for a simultaneous incorporation both of a fluid irrigation network and lesion assessment components configured to produce high-resolution three-dimensional real-time imagery of lesions during and/or after their formation.

Some embodiments provide for an irrigated catheter including an ablation electrode, at least one imaging device and an imaging device steering portion coupled to the at least one imaging device and configured to rotate the at least one imaging device in order to produce one or more images of lesions during and/or after their formation. The imaging device steering portion occupies a central region of the ablation electrode. The irrigated catheter further comprises a fluid network occupying a peripheral rather than a central region of the ablation electrode and, as such, at least a portion of the fluid network occupies a catheter peripheral region of that surrounds the imaging device steering portion. In some embodiments, the catheter peripheral region may surround an entire circumference of the imaging device steering portion.

As used herein, unless indicated otherwise by the context, the term approximately is generally understood to mean, for example, within 15%, within 10%, or within 5%, although one of skill would appreciate there is latitude in such numbers. As used herein, unless indicated otherwise by the context, the term “substantially” is understood to mean, for example, within 5%, within 3%, within 2%, or exactly, although one of skill would appreciate there is latitude in such numbers.

In this description, various aspects and features of the present invention will be described. The various features of the invention are discussed separately for clarity. One skilled in the art will appreciate that the features may be selectively combined in a device depending upon the particular application. Furthermore, any of the various features may be incorporated in a catheter and associated method of use for either mapping and/or ablation procedures.

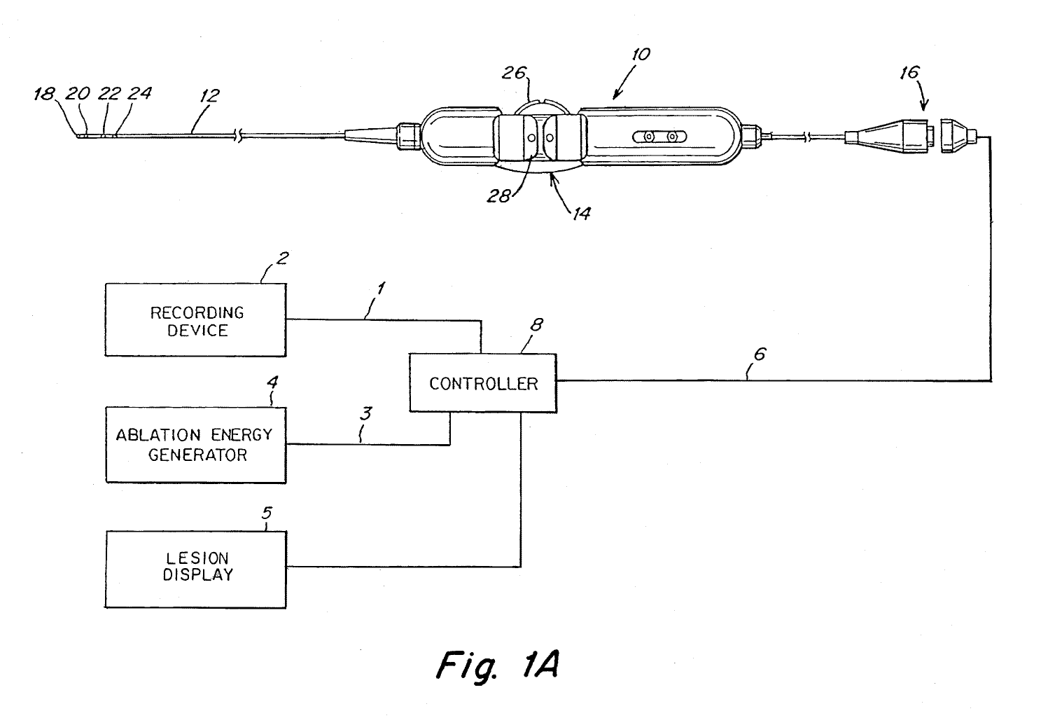

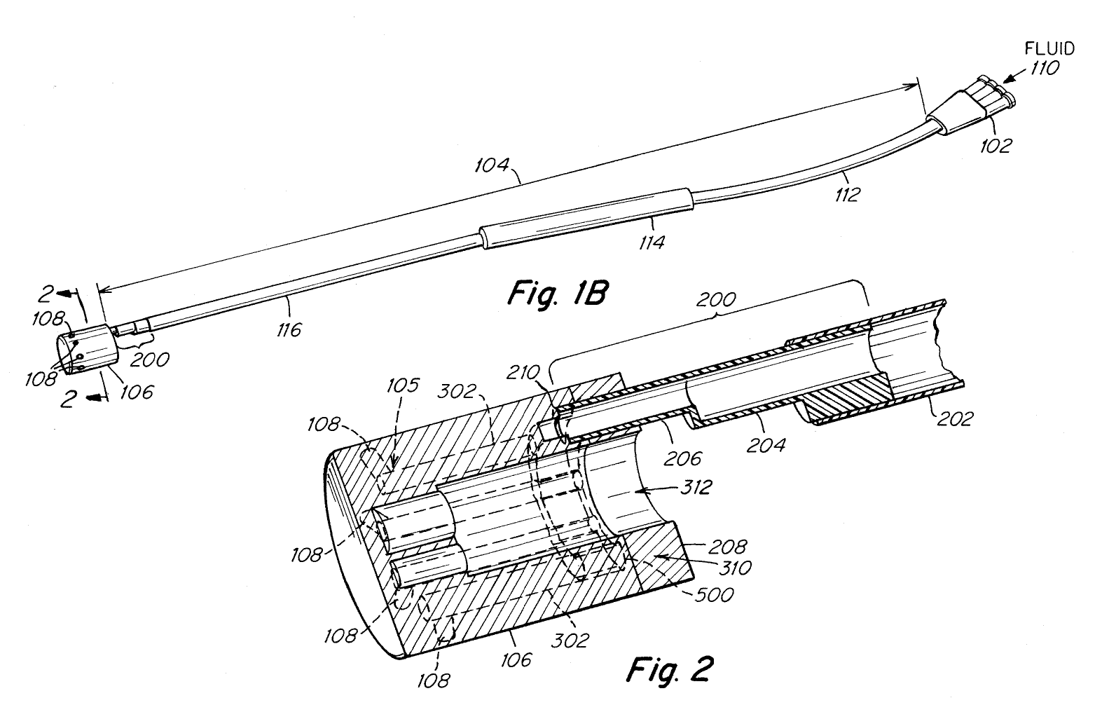

Reference is now made to FIG. 1A, which illustrates an overview of a mapping and/or ablation catheter system in accordance with one embodiment of the present invention. The system includes a catheter 10 having a shaft portion 12, a control handle 14, a connector portion 16, and electrodes 18, 20, 22, and 24. Control handle 14 may include actuation elements, such as a thumb wheel 26 or a slider 28, for bending segments of shaft portion 12. A controller 8 is connected to connector portion 16 via cable 6. Ablation energy generator 4 may be connected to controller 8 via cable 3. A recording device 2 may be connected to controller 8 via cable 1. A lesion display 5 may be connected to controller 8. Lesion display 5 may be configured to display imagery obtained at least in part by using one or more imaging devices (e.g., ultrasound, optical, etc.) in the irrigated catheter. When used in an ablation application, controller 8 is used to control ablation energy provided to catheter 10 by ablation energy generator 4. When used in a mapping application, controller 8 is used to process signals coming from catheter 10 and to provide these signals to recording device 2. Although illustrated as separate devices, recording device 2, ablation energy generator 4, and controller 8 could be incorporated into a single device or two devices.

FIG. 1B illustrates one embodiment of an irrigated catheter 100 including a fluid network 101 and an ablation electrode 106. Fluid network 101 has a fluid source 102 and a proximal longitudinal channel 104 fluidly coupled to fluid source 102. Fluid network 101 further includes a distal cooling portion 105 at least partially disposed within ablation electrode 106, and fluidly coupled to a proximal longitudinal channel 104. Distal cooling portion 105 comprises a plurality of channels configured to conduct fluid from the proximal longitudinal channel 104 through ablation electrode 106. Fluid network 101 further includes exit openings 108, defined in an exterior wall of the ablation electrode, from which fluid may be released to promote convective cooling of the electrode and/or to control temperature at the electrode-tissue interface. A fluid 110, such as saline, may enter fluid network 101 via fluid source 102, flow along the length of proximal longitudinal channel 104 into channels in the distal cooling portion 105 at least partially disposed within ablation electrode 106, and exit ablation electrode 106 via exit openings 108.

Proximal longitudinal channel 104 may have multiple sections including interface tubing 112 fluidly coupled (e.g., via a Luer fitting or in any other suitable way) to fluid source 102, transition tubing 114 fluidly coupled to interface tubing 112, irrigation line 116 fluidly coupled to transition tubing 114, and nozzle section 200 fluidly coupled to irrigation line 116. A fluid 110 may flow along the length of the proximal longitudinal channel 104 by flowing from fluid source 102 via interface tubing 112, via transition tubing 114, via irrigation line 116, and then via nozzle section 200 into ablation electrode 106. As shown, proximal longitudinal channel 104 comprises four sections, but in other embodiments a proximal longitudinal channel may comprise any suitable number of sections (e.g., one, at least two, at least three, at least five, at least ten, etc.), as aspects of the disclosure provided herein are not limited in this respect. For example, in some embodiments, a proximal longitudinal section may comprise a nozzle section and a line section fluidly coupled to the nozzle section and a fluid source so as to conduct fluid from the fluid source to the nozzle section.

The cross-sectional area of proximal longitudinal channel 104 may vary along its length to change the velocity of fluid flow along its length. In some embodiments, the cross-sectional area of proximal longitudinal channel 104 may decrease along the length of the channel from its proximal end toward its distal end, which may accelerate the flow of fluid along the length of proximal longitudinal channel 106. In this way, the velocity of the fluid entering irrigated ablation catheter 106 may be greater than the velocity of the fluid entering proximal longitudinal channel 104. In addition, the pressure drop along the proximal longitudinal channel may be limited.

The cross-sectional area of proximal longitudinal channel 104 may decrease in steps rather than gradually. In embodiments where a proximal longitudinal channel comprises multiple sections, the sections may have successively decreasing cross-sectional areas. For example, in the embodiment illustrated in FIG. 1B, transition tubing 114 has a smaller cross-sectional area than that of interface tubing 112, and irrigation line 116 has a smaller cross-sectional area than that of transition tubing 114. As previously described, the proximal longitudinal channel may comprise any suitable number of sections each having different cross-sectional areas. Accordingly, the cross-sectional area of the proximal longitudinal channel may decrease in any suitable number of steps, as aspects of the disclosure provided herein are not limited in this respect. In other embodiments, the cross-sectional area of proximal longitudinal section 104 may taper gradually rather than in a step-wise manner. For example, in some embodiments, the proximal longitudinal channel may comprise a nozzle section and a line section having a gradually tapered cross-sectional area and configured to conduct fluid from the fluid source to the nozzle section.

FIG. 2 illustrates a cutaway view of nozzle section 200 and ablation electrode 106 of the fluid network 100 shown in FIG. 1. Nozzle section 200 is fluidly coupled to channels in distal cooling portion 105, which comprises circumferential channel 500 at least partially disposed within ablation electrode 106. As shown, nozzle section 200 is fluidly coupled to circumferential channel 500 and is configured to conduct fluid from proximal longitudinal channel 104 to circumferential channel 500. Ablation electrode 106 comprises cover 208, and nozzle 200 is at least partially disposed within cover 208.

Nozzle section 200 may be configured to accelerate the flow of the fluid from proximal longitudinal channel 104 toward and into circumferential channel 500 so that the velocity of the fluid entering circumferential channel 500 is greater than the velocity of the fluid entering nozzle section 200. The increased fluid velocity in turn may allow for an even distribution of fluid through circumferential channel 500. The fluid velocity may be increased in any suitable manner. For example, a nozzle section may comprise multiple subsections having successively decreasing cross-sectional areas in order to accelerate the flow of fluid through the nozzle section. For example, in the illustrated embodiment, nozzle section 200 includes multiple subsections of successively decreasing cross-sectional areas. In particular, nozzle section 200 comprises nozzle subsection 202, nozzle subsection 204 having a cross-sectional area smaller than that of nozzle subsection 202, and nozzle subsection 206, having a cross-sectional area smaller than that of nozzle subsection 204. The decreasing cross-sectional areas of nozzle subsections 202, 204, and 206 cause the fluid velocity to increase as the fluid moves through nozzle section 200. As shown, nozzle section 200 comprises three subsections, but in other embodiments a nozzle section may comprise any other suitable number of subsections (e.g., one, two, at least four, at least five, at least ten, etc.), as aspects of the disclosure provided herein are not limited in this respect. It should also be appreciated that, in some embodiments, the nozzle section may comprise a single section having a gradually tapered cross-sectional area to accelerate the velocity of the fluid flowing through the nozzle section.

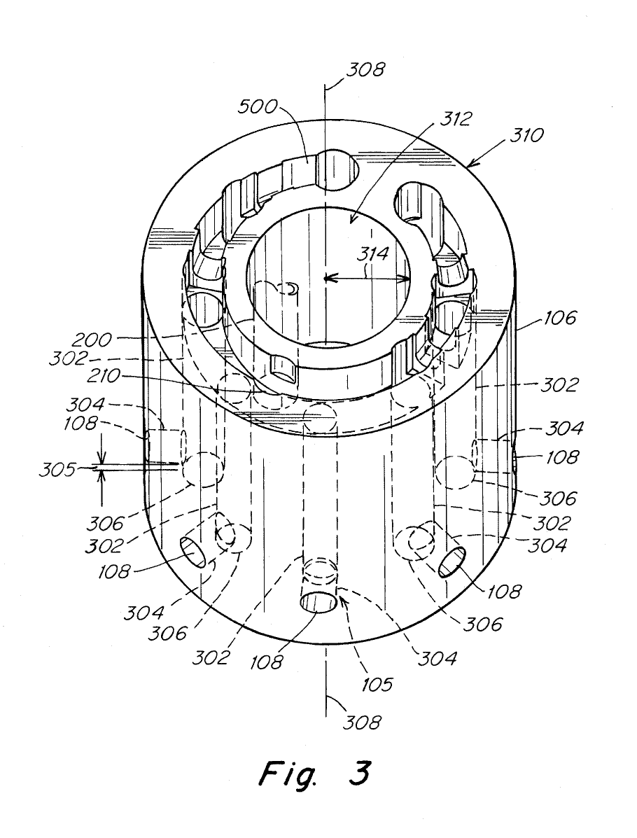

As previously described, nozzle section 200 is configured to conduct fluid into distal cooling portion 105, which are at least partially disposed within ablation electrode 106. As shown in FIG. 3, distal cooling portion 105 comprises circumferential channel 500 configured to conduct fluid about at least a portion of the circumference of ablation electrode 106, distal longitudinal channels 302 configured to conduct fluid along a distal length of ablation electrode 106, and radial channels 304 configured to conduct fluid between distal longitudinal channels 302 and exit openings 108.

Circumferential channel 500 comprises multiple openings (e.g., openings 502a-502d described below with reference to FIG. 5) that permit fluid to flow from circumferential channel 500 to distal longitudinal channels 302. As shown in FIG. 3, each of distal longitudinal channels 302 is fluidly coupled to circumferential channel 500 via a respective opening of circumferential channel 500.

Distal longitudinal channels 302 may include any suitable number of distal longitudinal channels. In some embodiments, the number of distal longitudinal channels is such that the channels may be symmetrically arranged about a region of circumferential channel 500. As one example, the number of distal longitudinal channels may be such that the channels are symmetrically disposed about opening 210 via which nozzle section 200 is fluidly coupled to circumferential channel 500. For example, distal longitudinal channels 302 may have an even number of channels (e.g., two, four, six, eight, ten, twelve, fourteen, sixteen, etc.). Though, in other embodiments, distal longitudinal channels 302 may have an odd number of channels (e.g., three, five, seven, nine, eleven, thirteen, fifteen, etc.), as aspects of the disclosure provided herein are not limited in this respect.

In some embodiments, the cross-sectional area of a particular distal longitudinal channel may be uniform along the length of the particular distal longitudinal channel. Though it should be appreciated that in such embodiments, the cross-sectional areas of different distal longitudinal channels need not be the same. For example, cross-sectional areas of two different distal longitudinal areas may be different from one another. In other embodiments, the cross-sectional area of a particular distal longitudinal channel may vary along its length.

To permit fluid to be released from the ablation electrode, each of distal longitudinal channels 302 is fluidly coupled to one or multiple radial channels configured to conduct fluid to one or more exit openings 108 disposed in an exterior wall of the ablation electrode. Each radial channel may have uniform or varying cross-sectional area. As shown in FIG. 3, each distal longitudinal channel 302 is fluidly coupled to a radial channel 304 allowing fluid to flow in a radial direction away from the distal longitudinal channel. The radial direction may be at any suitable angle to the distal longitudinal channel. For example, the radial direction may be at an angle of 90 degrees (i.e., perpendicular) to the distal longitudinal channel, at any angle in the range of 75-90 degrees to the distal longitudinal channel, at any angle in the range of 60-75 degrees to the distal longitudinal channel, at any angle in the range of 45-60 degrees to the distal longitudinal channel, at any angle in the range of 30-45 degrees to the distal longitudinal channel, or at any angle in the range of 5-30 degrees to the distal longitudinal channel. It should be appreciated that a distal longitudinal channel may be coupled to any suitable number of radial channels (e.g., at least two, at least four, at least eight, at least sixteen, etc.). In embodiments where a distal longitudinal channel is coupled to multiple radial channels, each distal longitudinal channel may be coupled to the same number of radial channels so that fluid is distributed uniformly about and released uniformly from ablation electrode 106.

At its proximal end, a distal longitudinal channel is fluidly coupled to circumferential channel 500. In some embodiments, a distal longitudinal channel may be fluidly coupled to a radial channel at a distance before the distal end of the distal longitudinal channel. In such embodiments, the distal longitudinal channel extends for that distance past the point at which it is coupled to the radial channel. For example, as shown in FIG. 3, a distal longitudinal channel is fluidly coupled to a radial channel at a distance 305 from the distal end of the distal longitudinal channel. In other embodiments, however, a distal longitudinal channel may turn into, rather than extend past, a radial channel, as aspects of the disclosure provided herein are not limited in this respect.

Although exit openings 108 are shown as having a circular shape, these openings may alternatively be semi-circular, linear, oval, or have any other suitable shape, as aspects of the disclosure provided herein are not limited in this respect. In addition, any suitable number of openings may be disposed within the exterior wall of ablation electrode 106.

Distal cooling portion 105 may occupy a region of ablation electrode peripheral to the central longitudinal axis of ablation electrode 106. In this way, other components of the catheter (e.g., sensors, wires, etc.) may be disposed within a central region of the ablation electrode, as described in greater detail below. For example, as shown in FIG. 3, channels in the distal cooling portion 105 (including circumferential channel 500, distal longitudinal channels 302, radial channels 304, and exit openings 108) occupy region 310 peripheral to central longitudinal axis 308 of ablation electrode 106. Peripheral region 310 is located at least a distance 314 away from central longitudinal axis 308 so that other components (e.g., one or more imaging devices, steering for the imaging device(s), etc.) of the catheter may be disposed within central region 312 of ablation electrode 106. As previously described, since a portion of a fluid network 101 (e.g., channels 105) is disposed within a peripheral (rather than a central) region of the ablation electrode, flow of fluid through the fluid network may cool the electrode (e.g., the exterior wall(s) of the electrode) along its length and may cool blood (or any other material such as tissue) adjacent to the exterior wall(s) of the electrode.

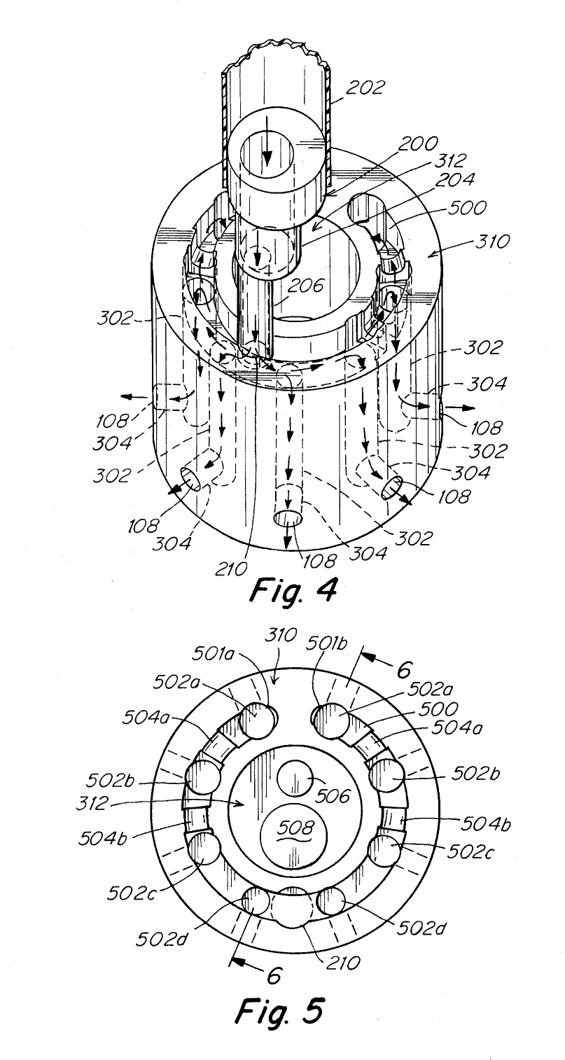

The flow of fluid through a portion of the fluid network is further illustrated in FIG. 4, which shows fluid flowing through nozzle section 200 (via subsections 202, 204, and 206) and opening 210 to circumferential channel 500. The fluid flow splits into two streams flowing along arms of circumferential channel 500. Each of the fluid streams further divides into multiple fluid streams following paths provided by distal longitudinal channels 304. The fluid streams follow distal longitudinal channels 302, enters radial channels 304 fluidly coupled to distal longitudinal channels 302, and exits the ablation electrode via exit openings 108.

In some embodiments, the fluid network may be constructed such that fluid (e.g., saline) exits the ablation electrode via exit openings 108 at velocities that allow the exiting fluid to quickly carry heat away from the ablation electrode. Releasing fluid from exit openings 108 at high velocities may help to control the temperature of the blood by efficiently carrying heat away from the source of energy (i.e., the ablation electrode) before the blood gets too hot. In some embodiments, fluid exits the irrigation ablation electrode at velocities in the range of 900-1600 mm/sec, 1000-1500 mm/sec, 1100-1500 mm/sec, 1100-1400 mm/sec, 1100-1300 mm/sec, 1200-1400 mm/sec, 1100-1300 mm/sec, or any other suitable range. In some embodiments, fluid exits the irrigation ablation electrode at velocities above 900 mm/sec, 1000 mm/sec, 1100 mm/sec, 1200 mm/sec, 1300 mm/sec, 1400 mm/sec, 1500 mm/sec, 1600 mm/sec, etc.

In some embodiments, the fluid network of a catheter may be constructed such that fluid conducted along proximal longitudinal channel 104 exits each of exit openings 108 at approximately the same velocity. In some embodiments, the fluid network may be constructed such that fluid conducted through the proximal longitudinal channel 104 exits each of exit openings 108 at substantially the same velocity. In particular, the fluid network (e.g., proximal longitudinal channel 104, nozzle section 200, circumferential channel 500, distal longitudinal channels 302, radial channels 304, exit openings 108, etc.) may be constructed and arranged so as to obtain approximately and/or substantially the same exit velocities of fluid at exit openings 108. Some aspects of the construction of circumferential channel 500 that lead to approximately and/or substantially the same exit velocities of fluid at exit openings 108 are described in further detail below.

FIG. 5 illustrates circumferential channel 500. Circumferential channel 500 may have an annular shape and, in some embodiments including the embodiment illustrated in FIG. 5, circumferential channel 500 may have a truncated annular shape such that the circumferential channel has ends 501a and 501b. Ends 501a and 501b may increase the pressure and the velocity of the fluid at exit openings 108 and may help to achieve approximately and/or substantially uniform fluid exit velocities at exit openings 108.

Circumferential channel 500 comprises multiple channel openings that allow fluid to flow from circumferential channel 500 to distal longitudinal channels 302. In the illustrated embodiment, circumferential channel 500 comprises channel openings 502a adjacent to ends 501a and 501b, channel openings 502b located between channel openings 502a and opening 210 (via which fluid enters circumferential channel 500 from nozzle section 200), channel openings 502c located between channel openings 502b and opening 210, and channel openings 502d located between channel openings 502c and opening 210. Although eight channel openings are illustrated in FIG. 5, it should be appreciated that circumferential channel may have any suitable number of channel openings (e.g., a channel opening for each distal longitudinal channel of which there may be any suitable number as previously described).

In some embodiments, channel openings of a circumferential channel may be symmetrically arranged about a region of the circumferential channel. For example, as illustrated in FIG. 5, channel openings of circumferential channel 500 may be arranged symmetrically about opening 210 via which nozzle section 200 is fluidly coupled to circumferential channel 500. Accordingly, circumferential channel 500 may comprise the same number of channel openings arranged between opening 210 and end 501a as the number of channel openings arranged between opening 210 and end 501b.

In some embodiments, there may be an even number of channel openings and these channel openings may be arranged in pairs, with each pair of channel openings being located at substantially the same distance from opening 210. For example, in the illustrated embodiments, each of channel openings 502a is located at substantially the same distance from opening 210. As another example, each of channel openings 502b is located at substantially the same distance from opening 210. As yet another example, each of channel openings 502c is located at substantially the same distance from opening 210. As yet another example, each of channel openings 502d is located at substantially the same distance from opening 210. Though, in some embodiments, there may be an odd number of channel openings.

In some embodiments, the diameters of the circumferential channel openings may vary in order to achieve approximately and/or substantially uniform exit velocities of fluid at exit openings 108. In some embodiments, the diameters of the circumferential channel openings may be proportional to the distance of the channel openings from opening 210 so that the farther the channel openings are from opening 210, the larger their respective diameters. For example, the diameters of channel openings 502a may be larger than the diameters of channel openings 502b. In turn, diameters of channel openings 502b may be larger than the diameters of channel openings 502c. In turn, diameters of channel openings 502c may be larger than the diameters of channel openings 502d.

It should be appreciated that although openings 502a-d are shown as having a circular shape, these openings may alternatively be semi-circular, linear, oval, or have any other suitable shape, as aspects of the disclosure provided herein are not limited in this respect. In such instances, the cross-sectional area of the openings may vary in accordance with the distance of the openings to opening 210 so that channel openings farther away from opening 210 (and closer to ends 501a and 501b) may have larger cross-sectional areas.

Circumferential channel 500 further comprises baffles to achieve approximately and/or substantially uniform exit velocities of fluid at exit openings 108. As shown, circumferential channel 500 comprises two baffles 504a located between channel openings 502a and 502b. Baffles 504a may be configured to deflect fluid flowing from proximal longitudinal channel 104 to flow into channel openings 502b. Circumferential channel 500 further includes two baffles 504b located between channel openings 502b and 502c. Baffles 504b may be configured to deflect fluid flowing from proximal longitudinal channel 104 to flow into channel openings 502c. Although four baffles are illustrated in FIG. 5, it should be appreciated that in other embodiments a circumferential channel may comprise any suitable number of baffles (e.g., zero, two, six, eight, ten, twelve, etc.). Baffles may be symmetrically arranged about a region of the circumferential channel. For example, baffles of circumferential channel 500 are arranged symmetrically about opening 210.

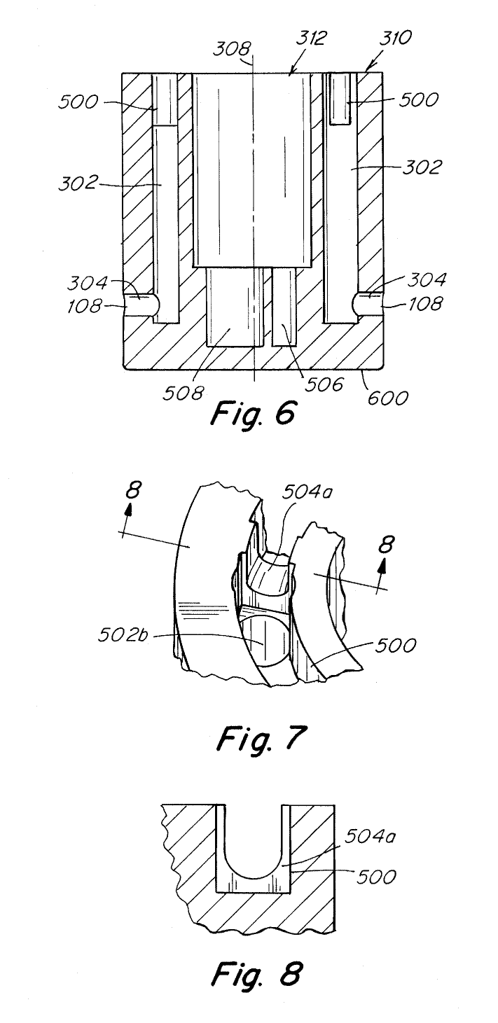

A baffle may reduce the amount and/or velocity of fluid flowing in the direction that the fluid is flowing. The impinging of fluid flow on the baffle may redirect the fluid flow and create a regional increase in pressure. A baffle may be constructed in any suitable way. For example, in some embodiments, a baffle may be a half wall and may comprise a saddle shaped section as shown in FIGS. 7 and 8. Though, a baffle may have any other suitable shape, as aspects of the disclosure provided herein are not limited in this respect.

As previously described, the arrangement of a catheters fluid network (e.g., distal portion 105) in a peripheral region of ablation electrode 106 (e.g., region 310) creates space in a central region (e.g., central region 312) of the ablation electrode that may be occupied by other components of a catheter. In some embodiments, region 312 may be occupied by one or more imaging devices used to obtain data for one or more images of the area around the ablation electrode and/or to assess one or more regions to which the ablation electrode has applied, is applying, and/or is to apply energy. As such, the imaging device(s) may be used to perform lesion assessment and/or any other suitable functions. For example, as illustrated in FIG. 5, ultrasound device 506 and optical coherence tomography device 508 occupy central region 312 of ablation electrode 106. Ultrasound device 506 may be any suitable type of ultrasound transducer configured to generate and sense ultrasound signals and may be configured to rotate about the central longitudinal axis (e.g., axis 308) of ablation electrode 106 in order to obtain data used to generate one or more ultrasound images. Optical coherence tomography device 508 may be any suitable device configured to generate a coherent radiation source and sense signals, and also may be configured to rotate about the central longitudinal axis (e.g., axis 308) to obtain data used to generate one or more tomographic images. Ultrasound device 506 and optical coherence tomography device 508 may be coupled to at least one processor (not shown) configured to receive data obtained by devices 506 and 508 to generate one or more ultrasound and/or tomographic images. Although one ultrasound device and one optical coherence tomography device are shown in FIG. 5, it should be appreciated that in some embodiments, any suitable number of any suitable types of imaging devices may occupy central region 312 (e.g., one or multiple ultrasound devices, one or multiple optical coherence tomography or other optical devices, one or multiple temperature sensors, one or multiple infrared devices, one or multiple RF devices, etc.), as aspects of the disclosure provided herein are not limited by the type and/or number of imaging devices that may occupy central region 312.

In embodiments where ablation electrode comprises at least one ultrasound device (e.g., within central region 312), the ablation electrode may be constructed so as to allow ultrasound energy to be transmitted and received through the electrode (e.g., so as to allow ultrasound energy to be propagate from within central region 312 to outside the electrode and vice versa). For example, in some embodiments, ablation electrode may be constructed from a thermoplastic polymer such as polymethylpentene (e.g., TPX). In other embodiments, ablation electrode may be constructed from a plastic coated with a metal (e.g., sputter coated) sufficiently thin to allow ultrasound energy to propagate through the coat. Any other suitable approach may additionally or alternatively be used.

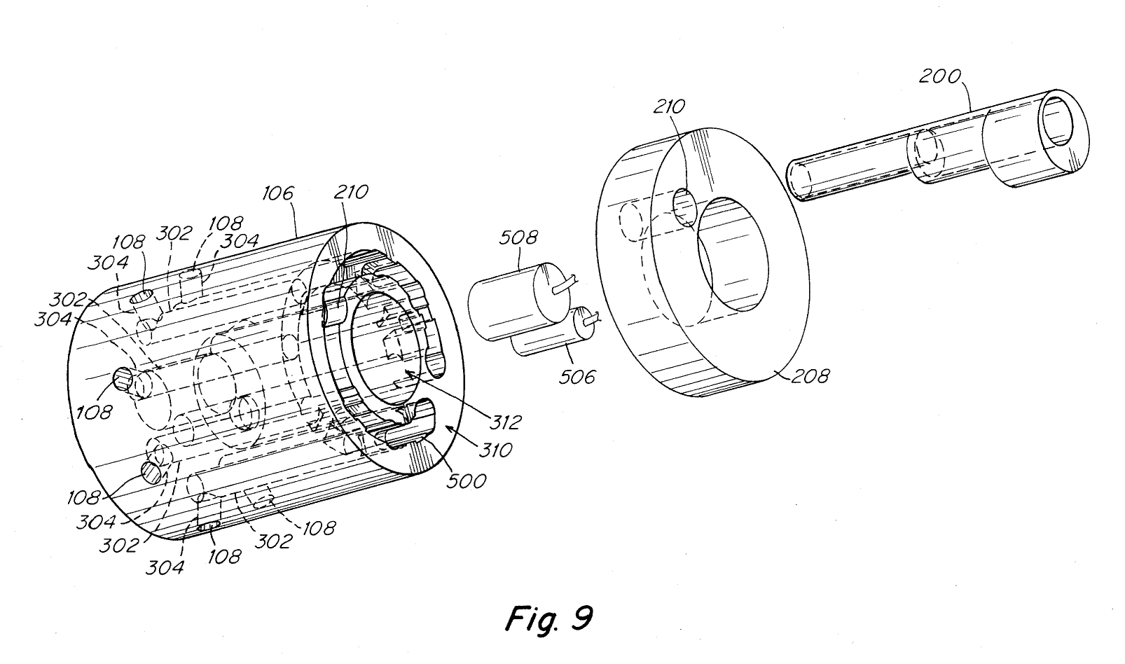

FIGS. 6 and 9 provide additional views of an embodiment of fluid network of an irrigated catheter. In particular, FIG. 6 is a cross-sectional view of ablation electrode 106 and shows the relative arrangement of distal longitudinal channels 302, radial channels 304, exit openings 108, ultrasound device 506, and optical coherent tomography device 508. Tip of ablation electrode 600 may be flat (as shown) or may be curved or any other suitable shape, as aspects of the disclosure provided herein are not limited in this respect.

FIG. 9 provides an exploded view of a fluid network of an irrigated catheter. In particular, FIG. 9 shows nozzle section 200, cover 208, and channels 105 comprising circumferential channel 500, distal longitudinal channels 302, radial channels 304, and exit openings 108. FIG. 9 also shows sections of ultrasound device 506 and optical coherence tomography device 508. As may be appreciated, circumferential channel 500 may cool ablation electrode 106 at the seam at which cover 208 is joined (e.g., welded) to ablation electrode 106.

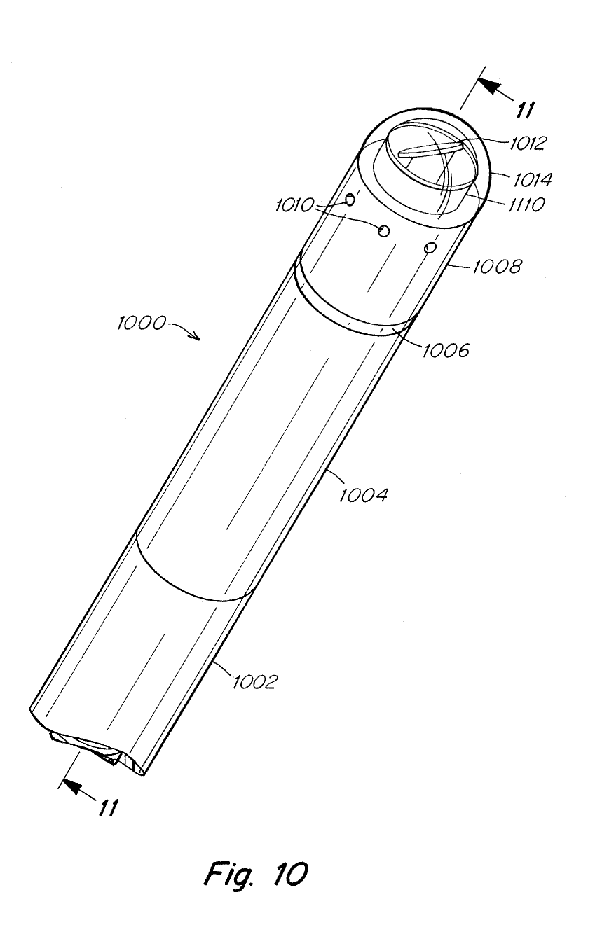

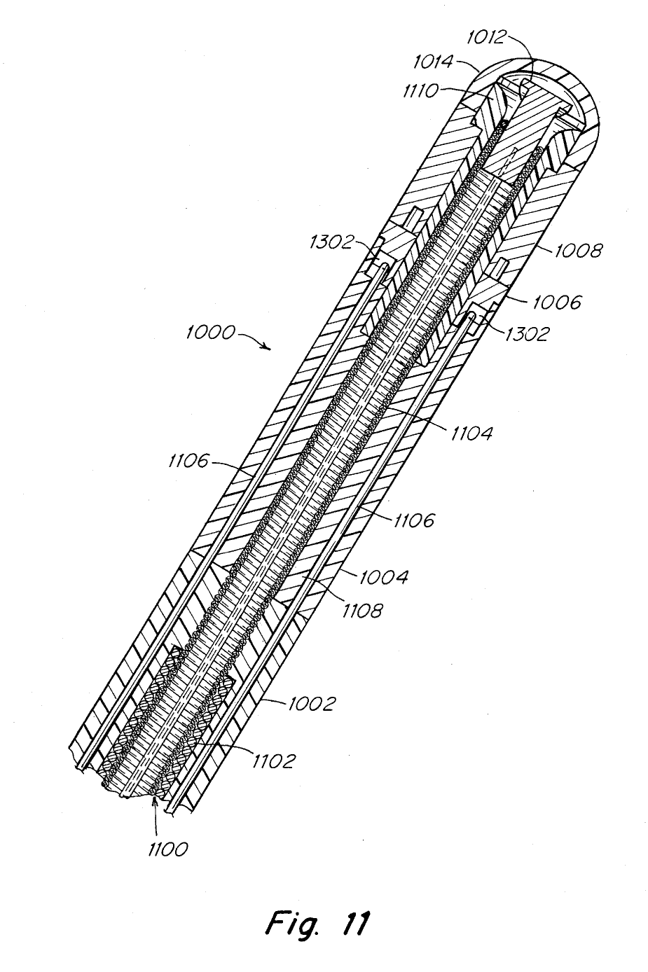

Another embodiment of an irrigated catheter is illustrated in FIG. 10, which is a perspective view of a distal portion of an irrigated catheter 1000. FIG. 11 is a cross-sectional view of the distal portion of the irrigated catheter 1000. The distal portion of catheter 1000 comprises a shaft portion 1002 coupled to a deflectable tip 1004, which is coupled to an ablation electrode 1008 via an interface 1006. Deflectable tip 1004 may be more flexible than shaft portion 1002 and may be controlled, via steering cables 1106, to bend (or move in any other suitable way) so as to bring the distal portion of the irrigated catheter 1000 to a desired configuration and/or position. As shown in FIG. 11, steering cables 1106 may be disposed a region in the jacket of the irrigated catheter 1000 that is peripheral to the central region of the catheter. The interface 1006 may be a stepped interface to help create a seal between the deflectable tip 1004 and the ablation electrode 1008.

Catheter 1000 comprises a fluid network at least partially disposed in the jacket of ablation electrode 1008. The fluid network comprises a plurality of channels (e.g., as previously described with reference to FIGS. 1B and 2-9) configured to conduct fluid (e.g., saline) along the length of the catheter to the ablation electrode, to conduct fluid throughout the ablation electrode, and to release the fluid from exit openings 1010 disposed in the wall of the ablation electrode. In this way, the fluid network is configured to conduct fluid throughout the catheter and release the fluid from the catheter to promote convective cooling of the ablation electrode and/or to control temperature at the electrode-tissue interface.

In some embodiments, the fluid network may comprise at least one proximal longitudinal channel configured to conduct fluid along a proximal length of the catheter, and a distal cooling portion configured to conduct fluid throughout the distal portion of the irrigated catheter. The distal cooling portion may be at least partially disposed in the jacket of ablation electrode 1008. The distal cooling portion may comprise a circumferential channel (e.g., circumferential channel 500) fluidly coupled to the at least one proximal longitudinal channel (e.g., proximal longitudinal channel 104). The circumferential channel may have an annular shape and, in some embodiments, may have a truncated annular shape. The distal cooling network may further comprise one or more distal longitudinal channels (e.g., distal longitudinal channels 302 shown in FIG. 3) fluidly coupled to the circumferential channel and configured to conduct fluid along a distal length of the catheter. Aspects of the fluid network, including alternative embodiments of the proximal longitudinal channel, are further described below.

The distal portion of catheter 1000 further comprises an imaging device 1012, which is covered by imaging device cover 1014. Imaging device cover may be attached to the catheter via a threaded interface. In some embodiments, imaging device 1012 may be an ultrasound imaging device having one or multiple ultrasound transducers. In embodiments in which imaging device 1012 comprises multiple ultrasound transducers, the ultrasound transducers may be arranged in an array and may be controlled to perform imaging jointly (e.g., via beamforming and/or other suitable imaging techniques). In other embodiments, imaging device 1012 may be an optical coherence tomography (OCT) imaging device comprising one or multiple OCT transducers. Though it should be appreciated that imaging device 1012 may be any other suitable type of imaging device including, but not limited to, the devices described with reference to FIG. 5, as aspects of the disclosure provided herein are not limited in this respect. In addition, although distal portion of catheter 1000 is shown as having only one imaging device, in some embodiments, a catheter may comprise multiple imaging devices (e.g., one or more ultrasound imaging devices and/or one or more OCT imaging devices).

In some embodiments, imaging device 1012 may be configured to direct energy about an angle of less than 180 degrees (e.g., less than 150 degrees, less than 120 degrees, less than 90 degrees, less than 60 degrees, less than 45 degrees, or less than 30 degrees). In such embodiments, including the embodiment shown in FIG. 10, at least a portion of the imaging device 1012 may be pitched at an angle to a longitudinal (e.g., the central longitudinal) axis of the catheter. It should be appreciated that imaging device 1012 is not limited to directing energy about a fixed angle and, in some embodiments, imaging device 1012 may be configured to direct energy to an adjustable range of angles. In some embodiments, the imaging device 1012 may be configured to image at least a portion of a lesion (e.g., one or more edges of a lesion, the entire lesion, a center portion of the lesion) as the lesion is being formed and/or after the lesion has been formed.

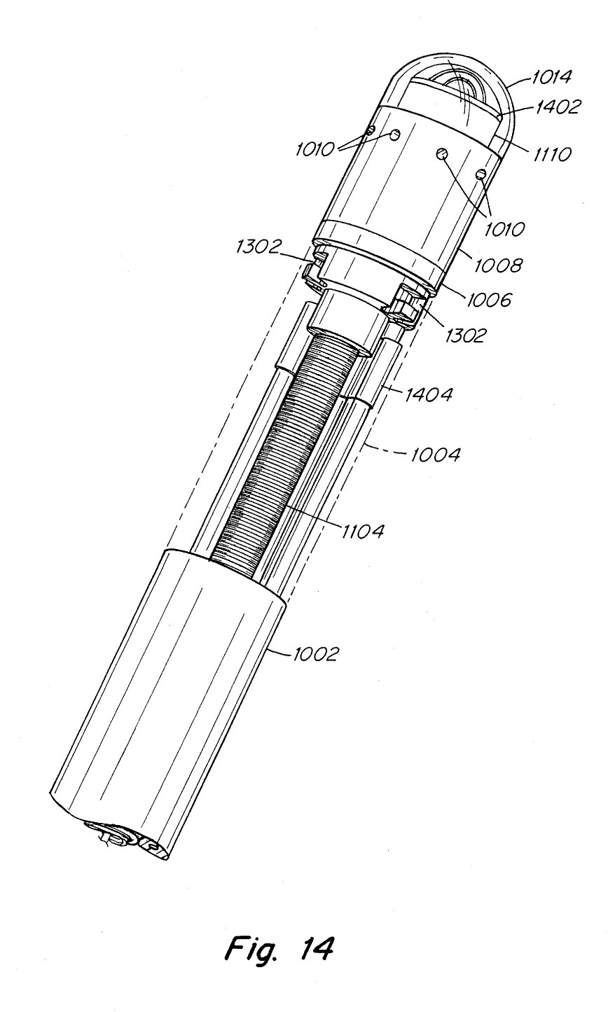

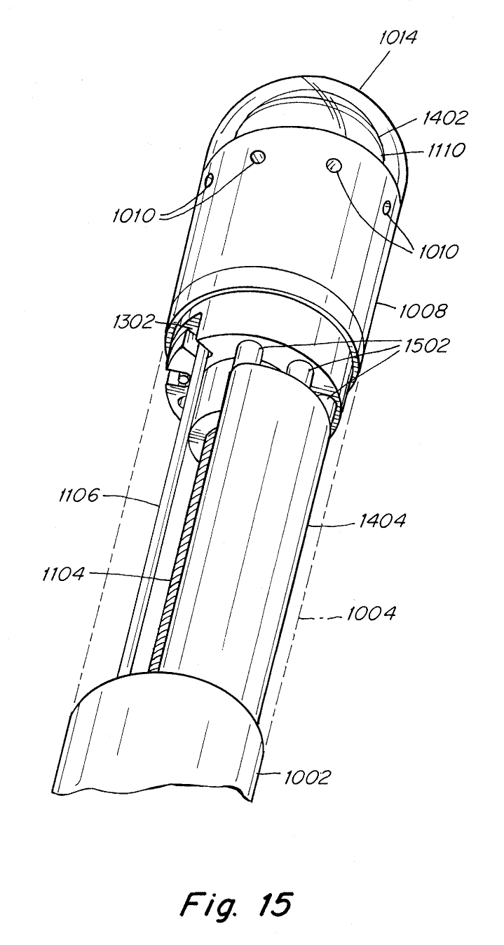

In some embodiments, such as the embodiments illustrated in FIGS. 14 and 15, the distal portion of catheter 100 may comprise an imaging device configured to direct energy about an angle of greater than 180 degrees. For example, as shown in FIGS. 14 and 15, the distal portion of catheter 1000 may comprise an imaging device (e.g., imaging device 1402) configured to direct energy to any suitable angle. An imaging device configured to direct energy about an angle of greater than 180 degrees may have any suitable shape and, for example, may be hemispherical.

As shown in FIG. 11, catheter 1000 further comprises an imaging device control portion 1100 coupled to imaging device 1012 and configured to rotate the imaging device 1012. The imaging device control portion 1100 comprises a steering column 1102 and a drive cable 1104 coupled to the steering column 1102. Steering column 1102 may strengthen shaft portion 1002 so that the shaft portion does not compress when it is bent, for example, when steering cables 1106 are used.

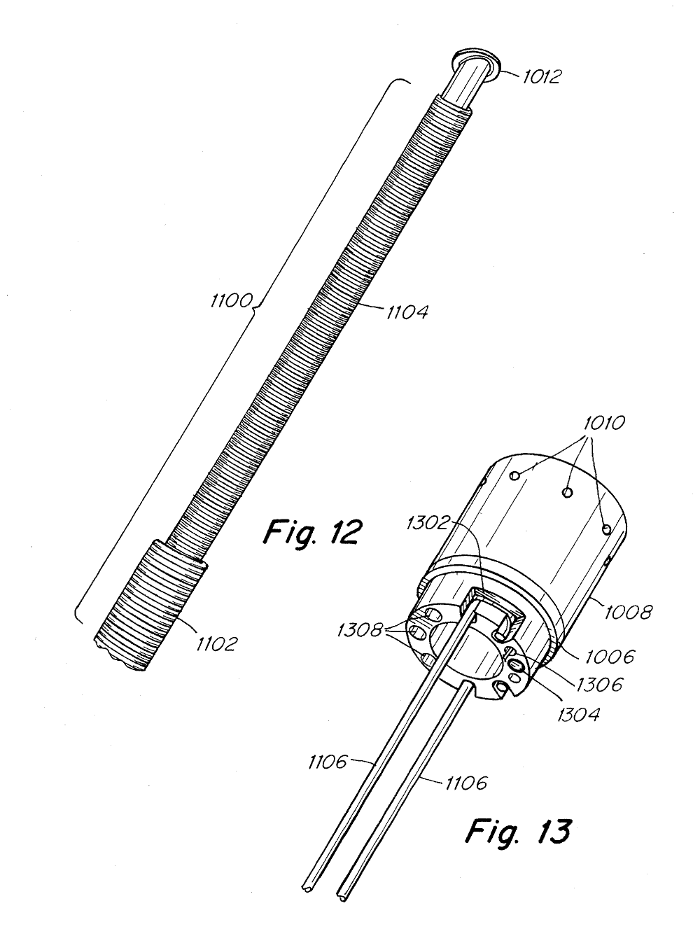

In the illustrated embodiment, the imaging device 1012 is coupled to the imaging device steering portion 1100 by being coupled to a distal end of the drive cable 1104. Shaft portion 1002 and deflectable tip 1004 are contoured to provide a bearing surface 1108 for the steering column 1102 and drive cable 1104. The bearing surface 1108 may prevent any translational movement of the steering column 1102 and drive cable 1104. Another view of an imaging device steering portion 1100 is shown in FIG. 12.

Drive cable 1104 may rotate in order to rotate imaging device 1012. In some embodiments, steering column 1102 may be affixed to drive cable 1104 and be configured to rotate together with drive cable 1104. In other embodiments, the drive cable 1104 may be configured to rotate independently of steering column 1102. For example, steering column 1102 may be affixed to shaft portion 1002 such that the steering column 1102 does not rotate when drive cable 1104 rotates.

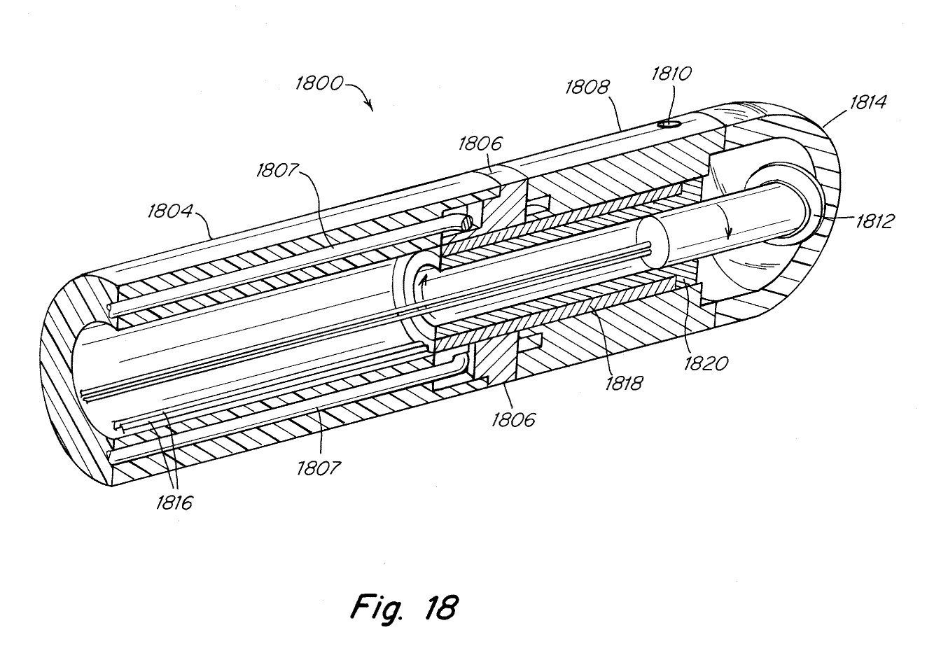

In some embodiments, rotation of the steering column 1102 and/or the drive cable 1104 may be driven by a proximally-placed motor (not shown). Accordingly, rotation of the imaging device 1012 may be driven by the proximally-placed motor. However, in some embodiments described below with reference to FIG. 18, rotation of an imaging device in an irrigated catheter may be driven by a distally-placed motor.

The imaging device control portion 1100 may be configured to rotate the imaging device 1012 at any suitable number of revolutions per minute. In some embodiments, the imaging device control portion may be configured to rotate the imaging device at any rate between 0 and 300 RPMs, at any rate between 600 and 2400 RPMs, at any rate between 800 and 2000 RPMs, at a rate of at least 100 RPMs, at a rate of at least 500 RPMs, at a rate of at least 1500 RPMs, and/or any other suitable rate.

In some embodiments, steering column 1102 may comprise a hollow tube. The tube may be a may be composed of any other suitable material(s) such as stainless steel, for example. The tube may be a cable tube, a braided tube, or any other suitable type of tube. In some embodiments, drive cable 1104 may comprise a torque transmission coil. The coil may be a round wire coil, a flat wire torque coil, or an inner lumen flat wire coil. The coil may be configured to transmit the rotation the proximal end of the coil to the distal tip of the coil. The coil may be composed of any suitable material or materials and, for example, may be a stainless-steel coil or a platinum and stainless steel coil.

As previously described, in some embodiments the fluid network of an irrigated catheter is at least partially disposed in a region of the ablation electrode peripheral to the central longitudinal axis of the ablation electrode. Because the fluid network occupies a peripheral rather than a central region of the ablation electrode, other components of the irrigated catheter may be disposed within the central region of the ablation electrode. For example, as shown in FIG. 11, the imaging device control portion 1100 is disposed within the central region of the ablation electrode 1008. As such, at least a portion of the fluid network occupies a catheter peripheral region that surrounds the imaging device steering portion. It should be appreciated that although the fluid network occupies a catheter peripheral region, the fluid network may not occupy the entire region. In some embodiments, the peripheral region may surround an entire circumference of the imaging device steering portion.

As previously described, the fluid network of an irrigated catheter may comprise a distal cooling portion at least partially disposed in the jacket of ablation electrode 1008. The distal cooling portion may surround at least a portion of a circumference (e.g., half the circumference or the entire circumference) of the imaging device steering portion 1100. For example, the distal cooling portion may surround at least a portion of steering column 1102 and/or drive cable 1104. The distal cooling portion may comprise a circumferential channel configured to conduct fluid about at least a part of a circumferential portion of the peripheral region. As such, the circumferential channel may at least partially surround the imaging device steering portion 1100.

Distal portion of catheter 1000 further comprises a reinforcing sleeve 1110 in the embodiment shown in FIG. 11. The reinforcing sleeve 1110 is disposed in a region between the distal cooling portion of the fluid network of catheter 1000 and image device control portion 1100.

In the embodiments described with reference to FIGS. 10-12, the imaging device was coupled to an image steering portion configured to rotate the imaging device. However, it should be appreciated that not all aspects of the disclosure provided herein are limited in this respect. For example, some embodiments provide for irrigated catheters comprising an imaging device shaft portion coupled to the at least one imaging device. In some embodiments, the imaging device shaft portion may comprise an imaging device steering portion (examples of which have been provided). In some embodiments, the imaging device shaft portion may occupy a central region of the catheter. Accordingly, the fluid network of an irrigated catheter may occupy a catheter peripheral region that surrounds the imaging device shaft portion.

In some embodiments, the imaging device shaft portion may be a sheath and/or a covering, and may be configured to house one or more electrical and/or optical links coupled to the at least one imaging device of the irrigated catheter. For example, the imaging device shaft portion may be configured to house one or more optical fibers coupled to the at least one imaging device, as the case may be when the imaging device comprises at least one optical coherence tomography transducer.

It should be appreciated that in embodiments in which an irrigated catheter comprises an imaging device steering portion, the imaging device may be steered (e.g., rotated). This may allow for obtaining three-dimensional imagery of a lesion with numerous types of imaging devices including, but not limited to, ultrasound and/or optical coherence tomography imaging devices. However, in embodiments in which an irrigated catheter does not comprise an imaging device steering portion, the at least one imaging device may be configured to perform imaging of lesions without being rotated. It should further be appreciated that, in some embodiments, the imaging device may be configured to perform imaging of lesions both when it is being rotated and when it is not being rotated. For example, when the imaging device is at an angle to the central longitudinal axis (as described above), the device may be rotated to provide for three-dimensional imaging of lesions.

FIG. 13 shows an illustrative example of how steering cables 1106 may be coupled to ablation electrode 1008. Ablation electrode 1008 comprises cavities 1302 used for securing steering cables 1106. In some embodiments, steering cables 1106 may be secured to ablation electrode 1008 at least in part by using a potting compound. In some embodiments, the steering cables 1106 may be bent in order to couple to ablation electrode 1108. As shown in FIG. 13, for example, steering cables 1106 may be bent to form “J-hooks” that hook onto the ablation electrode 1108 by using cavities 1302.

In some embodiments, the jacket of ablation electrode 1008 may include one or more longitudinal channels for guiding one or more components of the irrigated catheter along its length. For example, as shown in FIG. 13, ablation electrode 1008 comprises longitudinal channel 1304 for guiding a thermal sensor along the length of the catheter. Ablation electrode 1008 also comprises longitudinal channel 1306 for guiding a conductor wire along the length of the catheter. In the illustrated embodiment, the thermal sensor and conductor wire occupy a region of the catheter peripheral to the imaging device steering portion. It should be appreciated that the jacket of ablation electrode 1008 may comprise any suitable number (e.g., one, two, three, four, five, etc.) of longitudinal channels for guiding components of the irrigated catheter, as aspects of the disclosure provided herein are not limited in this respect.

As previously described, the fluid network of the irrigated catheter may comprise a distal cooling portion (e.g., distal cooling portion 105) and a proximal longitudinal channel fluidly coupled to the distal cooling portion. In some embodiments, the proximal longitudinal channel may be fluidly coupled to the distal cooling portion via a single opening (e.g., opening 210 described with reference to FIG. 2). In other embodiments, the proximal longitudinal channel may be fluidly coupled to the distal cooling portion via multiple (e.g., two, three, four, five, etc.) openings. For example, as shown in FIG. 13, ablation electrode 1008 comprises three openings 1308 for coupling the proximal longitudinal channel to the distal cooling component at least partially disposed in the ablation electrode.

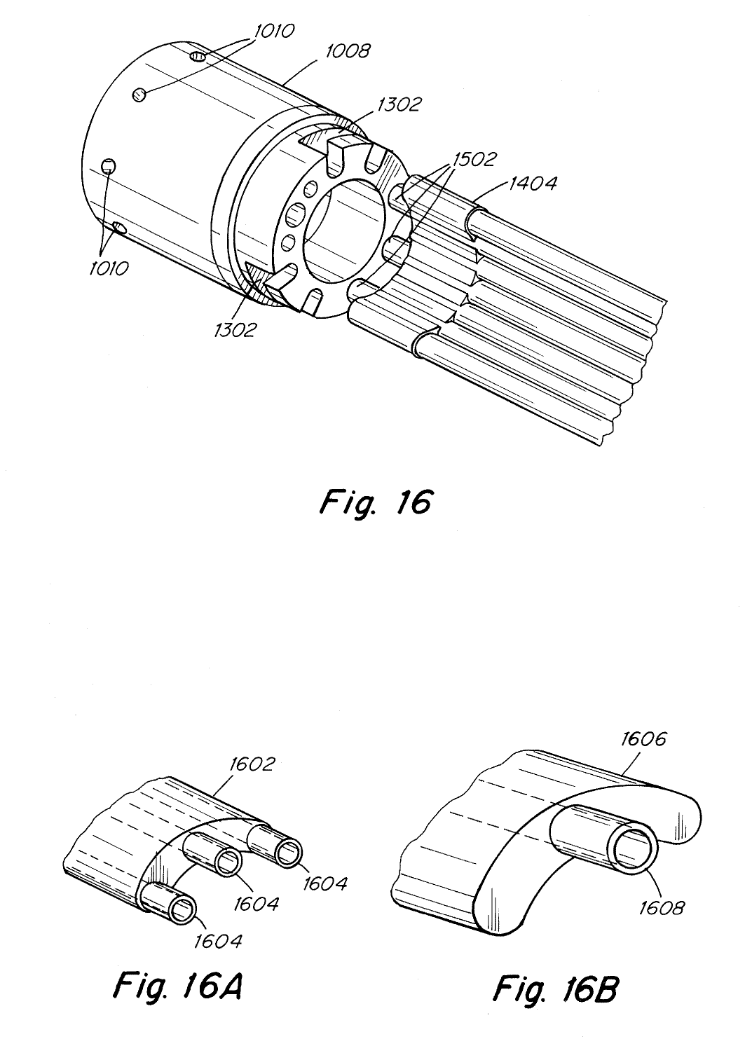

In some embodiments, the proximal longitudinal channel of an irrigated catheter's fluid network may comprise a contoured infusion line such as, for example, a contoured infusion line 1404 as illustrated in FIGS. 14, 15, and 16 which show different views of irrigated catheter 1000. The contoured infusion line may be configured to attach to one or more nozzles each of which may be fluidly coupled to distal cooling portion at least partially disposed in the ablation electrode. For example, as shown in FIG. 15, contoured infusion line 1404 is attached to three nozzles 1502, which are fluidly coupled to openings 1308. FIG. 16A shows another example of a contoured infusion line 1602 attached to three infusion nozzles 1604. FIG. 16B shows an infusion nozzle 1606 attached to a single infusion nozzle 1608.

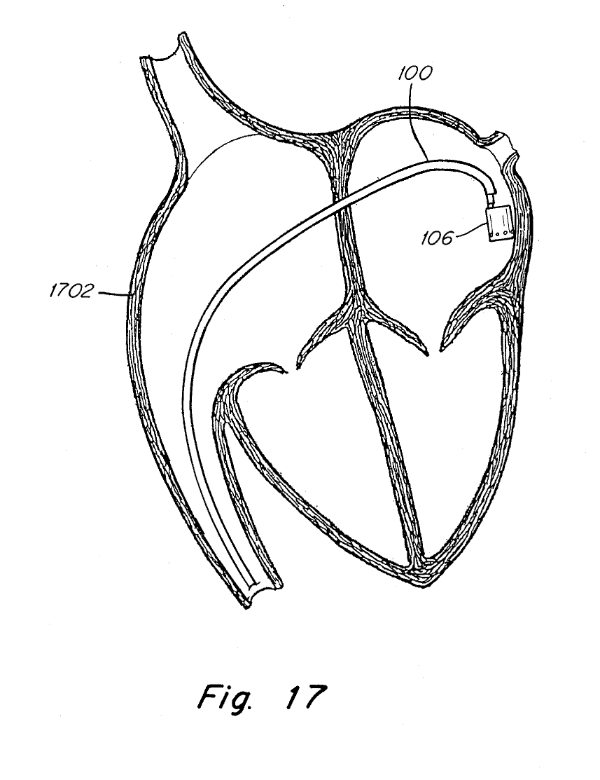

Reference is now made to FIG. 17, which illustrates how catheter 100, which has an ablation electrode, at least one imaging device, and a fluid network as described herein, may be used in endocardial applications. The catheter is introduced into a patient's heart 1702. Imaging guidance (e.g., direct visual assessment, camera port, fluoroscopy, echocardiography, magnetic resonance, ultrasound, optical coherence tomography, etc.) may be used to introduce the catheter into the heart. FIG. 17 in particular illustrates ablation electrode 106 introduced into the left atrium of the patient's heart although procedures may be performed in other chambers. Electrodes on the catheter may be used to sense signals in the heart to determine a desired location for ablation.

Once at a desired location in the heart 1702, the ablation electrode is configured so as to ablate the tissue adjacent to the catheter and apply energy to the adjacent tissue to form one or more lesions in the tissue. The fluid network of catheter 100 is used to conduct fluid through the catheter (e.g., through a peripheral region of the ablation electrode and out of exit openings disposed in the exterior wall of the ablation electrode) to cool the ablation electrode and to cool the blood and/or tissue adjacent to the ablation electrode. To determine whether a formed lesion is sufficient to cause a sufficient degree of conduction block, one or more imaging devices (e.g., one or more ultrasound sensors, one or more optical coherence tomography sensors, etc.) on the catheter may be used to assess the lesion(s).

In some embodiments, assessing the lesion may comprise imaging the lesion with at least one imaging device as the imaging device rotates. The imaging device may be rotated at least in part by using an imaging device steering portion, examples of which have been described herein. A lesion may be assessed while it is being formed and/or after it has been formed.

The lesion(s) formed in the manner described above may be used to treat arrhythmias (e.g., atrial fibrillation) in the heart and/or other heart conditions. It should also be appreciated that a catheter having a fluid network as described herein may be used in other applications. As one non-limiting example, the catheter may be used to form one or more lesions while performing renal denervation to treat arterial hypertension by partially reducing or completely blocking renal sympathetic nerve activity.

In some embodiments, such as the embodiments described with reference to FIGS. 10 and 11, rotation of an imaging device of an irrigated catheter may be driven by a proximally-placed motor and a drive cable that extends the length of the irrigated catheter. For example, rotation of imaging device 1012 may be driven by a proximally-placed motor and a drive cable 1104. The inventors have appreciated that, in some instances, a proximally-driven drive cable may stick or bind when the deflectable tip of the irrigated catheter is sufficiently bent. The deflectable tip of a catheter may be manipulated into one or more arcs of varying radii to navigate the catheter to various areas of interest within the heart. Such manipulation may generate catheter tip radii of curvature sufficiently small to induce the sticking or binding of the drive cable. This sticking or binding may lead to a non-uniform rotation of the imaging device(s) in the catheter and, in turn, result in non-uniform distortion of images obtained by the imaging device(s).

The inventors have recognized that rotation of the imaging device(s) may be driven by a distally-located motor, rather than by a proximally-located motor, to mitigate the problem of sticking or binding of a drive cable. Indeed, when a distally-located motor is used to drive rotation of the imaging device(s), there may not be a need to have a rotating drive cable that extends the length of the entire catheter.

Accordingly, some embodiments provide for an irrigated catheter having one or multiple imaging devices and a distally-located motor to drive rotation of the imaging device(s). One such embodiment is illustrated in FIG. 18, which is a cross-sectional view of a distal portion of an irrigated catheter 1800.

The distal portion of catheter 1800 comprises a shaft portion (not shown) coupled to a deflectable tip 1804, which is coupled to an ablation electrode 1808 via an interface 1806. Deflectable tip 1804 may be flexible and may be controlled, via steering cables 1807, to bend (or move in any other suitable way) so as to bring the distal portion of the irrigated catheter 1800 to a desired configuration and/or position. Steering cables 1807 may be disposed in a region of a jacket of the irrigated catheter 1800 that is peripheral to the central region of the catheter. The interface 1806 may be a stepped interface to help create a seal between the deflectable tip 1804 and the ablation electrode 1808.

Catheter 1800 comprises a fluid network at least partially disposed in the jacket of ablation electrode 1808. The fluid network comprises a plurality of channels (e.g., as previously described with reference to FIGS. 1B and 2-9) configured to conduct fluid (e.g., saline) along the length of the catheter to the ablation electrode, to conduct fluid throughout the ablation electrode, and to release the fluid from exit openings 1810 disposed in the wall of the ablation electrode. In this way, the fluid network is configured to conduct fluid throughout the catheter and configured to release the fluid from the catheter to promote convective cooling of the ablation electrode and/or to control temperature at the electrode-tissue interface. The fluid network may be of any suitable type described herein and, for example, may be the type of fluid network described with reference to FIG. 10.

The distal portion of catheter 1800 further comprises an imaging device 1812, which is covered by imaging device cover 1814. In some embodiments, imaging device 1812 may the same type of imaging device as imaging device 1012 (e.g., an ultrasound imaging device, an optical coherence tomography imaging device, etc.). Though it should be appreciated that imaging device 1812 may be any other suitable type of imaging device including, but not limited to, the devices described with reference to FIG. 5, as aspects of the disclosure provided herein are not limited in this respect. Although distal portion of catheter 1800 is shown as having only one imaging device, in some embodiments, a catheter may comprise multiple imaging devices.

As shown in FIG. 18, catheter 1800 further comprises a distally-located motor configured to rotate the imaging device 1812. The distally-located motor may occupy a central region of the irrigated catheter. Accordingly, the fluid network of the irrigated catheter may occupy a catheter peripheral region that surrounds the distally-located motor. The distally-located motor includes motor leads 1816 coupled to a stator 1818 (e.g., a coiled stator) that is attached to rotor 1820 (e.g., a cylindrical rotor).

In some embodiments, the distally-located motor may be an ultrasonic motor. The ultrasonic motor may rotate based on ultrasonic oscillations obtained from an ultrasonic oscillator. For example, ultrasonic waves may propagate from an external ultrasonic oscillator along motor leads 1816 to stator 1818 to rotate rotor 1829. Though, the distally located motor is not limited to being an ultrasonic motor and may be of any other suitable type (e.g., an electromagnetic motor). In some embodiments, the distally-located motor may have a diameter of less than 3 mm, less than 2 mm, less than 1 mm, less than 0.8 mm, or less than 0.5 mm.

The distally-located motor may be configured to rotate the imaging device 1812 at any suitable number of revolutions per minute. In some embodiments, the imaging device control portion may be configured to rotate the imaging device at any rate between 0 and 300 RPMs, at any rate between 600 and 2400 RPMs, at any rate between 800 and 2000 RPMs, at a rate of at least 100 RPMs, at a rate of at least 500 RPMs, at a rate of at least 1500 RPMs, and/or at any other suitable rate.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1-22. (canceled)

23. A catheter comprising:
an ablation electrode;
at least one imaging device;
an imaging device steering portion coupled to the at least one imaging device and configured to rotate the at least one imaging device;
a fluid network configured to conduct fluid along a length of the catheter and occupying a catheter peripheral region that surrounds the imaging device steering portion.

24. The catheter of claim 23, wherein the peripheral region surrounds an entire circumference of the imaging device steering portion.

25. The catheter of claim 23, wherein the fluid network comprises a distal cooling portion, wherein the distal cooling portion occupies the peripheral region of the catheter.

26. The catheter of claim 25, wherein the distal cooling portion comprises:
a circumferential channel configured to conduct fluid about at least a part of a circumferential portion of the peripheral region.

27. The catheter of claim 23, wherein the imaging device steering portion is configured to rotate the at least one imaging device at a rate in a range of 600 to 2400 revolutions per minute.

28. The catheter of claim 23, wherein the imaging device steering portion comprises a steering column and a drive cable coupled to the steering column, and wherein the at least one imaging device is coupled to a distal end of the drive cable.

29. The catheter of claim 28, further comprising:
a reinforcing sleeve at least partially disposed within the ablation electrode and provides a bearing surface to the drive cable.

30. The catheter of claim 28, wherein the drive cable is configured to rotate independently of the steering column.

31. The catheter of claim 28, wherein the drive cable configured to rotate together with the steering column.

32. The catheter of claim 23, wherein the at least one imaging device comprises an ultrasound transducer.

33. The catheter of claim 32, wherein the at least one imaging device comprises an array of ultrasound transducers.

34. The catheter of claim 23, wherein the at least one imaging device comprises an optical coherence tomography transducer.

35. The catheter of claim 23, wherein the at least one imaging device is configured to direct energy about an angle of less than 180 degrees.

36. The catheter of claim 23, wherein the at least one imaging device is configured to direct energy about an adjustable range of angles.

37. The catheter of claim 23, further comprising:
a shaft;
a deflectable tip coupled to the shaft; and
at least one steering cable configured to move the deflectable tip.

38. The catheter of claim 37, wherein the imaging device steering portion comprises a steering column and a drive cable, wherein the steering column is affixed to the shaft such that the drive cable rotates independently of the steering column.

39. The catheter of claim 23, further comprising a thermal sensor at least partially disposed in the ablation electrode and occupying a region of the catheter peripheral to the steering portion.

40. The catheter of claim 23, further comprising a conductor wire at least partially disposed in the ablation electrode and occupying a region of the catheter peripheral to the imaging device steering portion.

41. The catheter of claim 23, wherein the fluid network further comprises:
at least one proximal longitudinal channel configured to conduct fluid along a proximal length of the catheter; and
a distal cooling portion comprising:
a circumferential channel having an annular shape and fluidly coupled to the at least one proximal longitudinal channel, wherein the circumferential channel is configured to conduct fluid about at least a part of a circumferential portion of the catheter; and
a plurality of distal longitudinal channels fluidly coupled to the circumferential channel, the plurality of distal longitudinal channels configured to conduct fluid along a distal length of the catheter.

42. The catheter of claim 41, wherein the fluid network comprises the at least one proximal longitudinal channel, wherein the at least one proximal longitudinal channel comprises a nozzle section having at least one nozzle, the at least one nozzle having a stepped diameter.

43. The catheter of claim 42, wherein the at least one nozzle consists of three nozzles.

44. The catheter of claim 42, wherein the at least one nozzle consists of one nozzle.

45. The catheter of claim 41, wherein the circumferential channel has a truncated annular shape.

46. A method of using a catheter to treat tissue, the catheter comprising an ablation electrode, at least one imaging device, an imaging device steering portion coupled to the at least one imaging device and configured to rotate the at least one imaging device, and a fluid network configured to conduct fluid along a length of the catheter, the method comprising:
forming a lesion in the tissue using ablation energy emitted by the ablation electrode;
conducting fluid through the fluid network to cool the ablation electrode; and
imaging the lesion using the at least one imaging device,
wherein the fluid network occupies a catheter peripheral region that surrounds the imaging device steering portion.

47. The method of claim 46, wherein the peripheral region surrounds an entire circumference of the imaging device steering portion.

48. The method of claim 46, wherein the fluid network comprises a distal cooling portion, wherein the distal cooling portion occupies the peripheral region of the catheter.

49. The method of claim 46, wherein the distal cooling portion comprises:
a circumferential channel configured to conduct fluid about at least a part of a circumferential portion of the peripheral region.

50. The method of claim 46, wherein imaging the lesion comprises using the imaging device steering portion to rotate the at least one imaging device.

51. The method of claim 50, wherein using the imaging device steering portion to rotate the at least one imaging device comprises rotating the at least one imaging device at a rate in a range of 600 to 2400 revolutions per minute.

52. The method of claim 46, wherein imaging the lesion comprises imaging the lesion while the lesion is being formed.

53. A catheter comprising:
an ablation electrode;
at least one imaging device;
an imaging device shaft portion coupled to the at least one imaging device; and
a fluid network configured to conduct fluid along a length of the catheter and occupying a catheter peripheral region that surrounds the imaging device shaft portion.

54. The catheter of claim 53, wherein the imaging device shaft portion is configured to house one or more electrical and/or optical links coupled to the at least one imaging device.

55. The catheter of claim 54, wherein the imaging device shaft portion is configured to house one or more optical fibers coupled to the at least one imaging device.

56. The catheter of claim 55, wherein the at least one imaging device comprises an optical coherence tomography transducer.

57. The catheter of claim 56, wherein the imaging device shaft portion comprises an imaging device steering portion configured to rotate the at least one imaging device.
